(19) United States
(12) Patent Application Publication
(10) Pub. No.: US 2024/0326056 A1
(43) Pub. Date: Oct. 3, 2024

(54) MICROFLUIDIC CHIP, METHOD OF CONTROLLING FLOW VELOCITY OF FLUID, AND METHOD OF USING MICROFLUIDIC CHIP

(71) Applicants: BEIJING BOE TECHNOLOGY DEVELOPMENT CO., LTD., Beijing (CN); BOE TECHNOLOGY GROUP CO., LTD., Beijing (CN)

(72) Inventors: Fan Yang, Beijing (CN); Lin Deng, Beijing (CN); Ding Ding, Beijing (CN)

(21) Appl. No.: 18/579,414

(22) PCT Filed: May 20, 2022
(86) PCT No.: PCT/CN2022/094232

§ 371 Date: Jan. 15, 2024

(51) Int. Cl. B01L 3/00

(52) U.S. Cl. CPC B01L 3/502784; B01L 2200/0652; B01L 2300/0809; B01L 2300/0861

(57) ABSTRACT

The present disclosure provides a microfluidic chip, a method of controlling the flow velocity of a fluid in the microfluidic chip, and a method of using the microfluidic chip. The microfluidic chip includes at least one shunt structure, each shunt structure includes at least two channels each including a first channel and a second channel, the first channel is configured to allow the first fluid to flow therein, the second channel is configured to allow the second fluid to flow therein, and the first fluid and the second fluid merge at the first confluence of the microfluidic chip. The first channel has a first cross-section and a first length, the second channel has a second cross-section and a second length, the area of the first cross-section is greater than or equal to the area of the second cross-section, and the first length is less than or equal to second length.

### RELATED APPLICATION

This application is a 35 U.S.C. 371 national stage application of PCT International Application No. PCT/CN2022/094232 filed on May 20, 2022, the entire disclosure of which is incorporated herein by reference.

### TECHNICAL FIELD

The present disclosure relates to the field of biomedical detection, and in particular to a microfluidic chip, a method of controlling the flow velocity of a fluid in the microfluidic chip, and a method of using the microfluidic chip.

### BACKGROUND

Microfluidics is a technology that precisely controls and manipulates micro-scale fluids. With this technology, the basic operation units involved in the detection and analysis process, such as sample preparation, reaction, separation, and detection, can be integrated on a centimeter-scale chip. Microfluidics is generally used in the analysis process of trace drugs in the fields such as biology, chemistry, and medicine. The most common application of microfluidic droplets is as a micro-reactor to study reactions and processes at the microscale. Droplets as micro-reactors have attracted more and more attention due to their advantages such as small volume, no sample diffusion, stable reaction conditions, zero cross-contamination between samples, and rapid mixing.

### SUMMARY

According to an aspect of the present disclosure, a microfluidic chip is provided, and the microfluidic chip comprises at least one shunt structure. Each of the at least one shunt structure comprises at least two channels, the at least two channels comprise a first channel and a second channel, the first channel is configured to allow a first fluid to flow therein, the second channel is configured to allow a second fluid to flow therein, and the first fluid and the second fluid merge at a first confluence of the microfluidic chip. The first channel has a first cross-section and a first length, the second channel has a second cross-section and a second length, the first cross-section is perpendicular to a flow direction of the first fluid in the first channel, the second cross-section is perpendicular to a flow direction of the second fluid in the second channel, an area of the first cross-section is greater than or equal to an area of the second cross-section, and the first length is less than or equal to the second length.

In some embodiments, the area of the first cross-section is equal to the area of the second cross-section and the first length is less than the second length, and a relationship between a first ratio of a second flow velocity of the second channel to a first flow velocity of the first channel and a second ratio of the first length to the second length is substantially linear.

In some embodiments, a square value of the linear correlation coefficient between the first ratio and the second ratio is 0.9995.

In some embodiments, the first cross-section is in a shape of circular and has a first diameter, and the second cross-section is in a shape of circular and has a second diameter, the first length is equal to the second length and the first diameter is greater than the second diameter, and a relationship between a first ratio of a second flow velocity of the second channel to a first flow velocity of the first channel and a third ratio of a square of the second diameter to a square of the first diameter is substantially linear.

In some embodiments, a square value of the linear correlation coefficient between the first ratio and the third ratio is 0.9994.

In some embodiments, the area of the first cross-section is greater than the area of the second cross-section and the first length is less than the second length, the first channel and the second channel merge at the first confluence.

In some embodiments, the first cross-section has a first width in a first direction, the first direction is perpendicular to the flow direction of the first fluid in the first channel, the first fluid comprises droplets; the second cross-section has a second width in a second direction, the second direction is perpendicular to the flow direction of the second fluid in the second channel; the first width is larger than a particle size of each of the droplets, and the second width is smaller than the particle size of each of the droplets.

In some embodiments, a shape of the first channel and the second channel is arc.

In some embodiments, the area of the first cross-section is equal to the area of the second cross-section and the first length is less than the second length, the first channel and the second channel merge at the first confluence.

In some embodiments, the first channel comprises at least one section, each of the at least one section is in a shape of S-shaped, and the second channel comprises at least one section, each of the at least one section is in a shape of reverse S-shaped.

In some embodiments, a number of the sections of the second channel is the same as a number of the sections of the first channel, and a length of each section of the second channel is greater than a length of each section of the first channel.

In some embodiments, each shunt structure further comprises a third channel configured to allow the second fluid to flow therein, the first channel, the second channel and the third channel merge at the first confluence, the third channel has a third cross-section and a third length, the third cross-section is perpendicular to a flow direction of the second fluid in the third channel, the area of the first cross-section, the area of the second cross-section and an area of the third cross-section are equal, and the first length is less than the second length and the third length.

In some embodiments, the first channel is between the second channel and the third channel, and the second channel and the third channel are axisymmetric with respect to the first channel.

In some embodiments, each shunt structure further comprises: a third channel having a third cross-section and configured to allow a third fluid to flow therein, the third cross-section being perpendicular to a flow direction of the third fluid in the third channel; a fourth channel having a fourth cross-section and configured to allow a fourth fluid to flow therein, the fourth cross-section being perpendicular to a flow direction of the fourth fluid in the fourth channel, the third channel and the fourth channel merging at a second confluence of the microfluidic chip; and a connecting channel communicating with the first confluence and the second confluence respectively, the area of the first cross-section is greater than the area of the second cross-section, and an area of the third cross-section is greater than an area of the fourth cross-section.

In some embodiments, the third channel has a third length, the fourth channel has a fourth length, the first length is less than or equal to the second length, and the third length is less than or equal to the fourth length.

In some embodiments, the first cross-section has a first width in a first direction, the first direction is perpendicular to the flow direction of the first fluid in the first channel, the second cross-section has a second width in a second direction, the second direction is perpendicular to the flow direction of the second fluid in the second channel, the third cross-section has a third width in a third direction, the third direction is perpendicular to the flow direction of the third fluid in the third channel, the fourth cross-section has a fourth width in a fourth direction, the fourth direction is perpendicular to the flow direction of the fourth fluid in the fourth channel, the first fluid comprises a first type of droplets, the third fluid comprises a second type of droplets, the first width is greater than a particle size of each of the first type of droplets and the second width is less than the particle size of each of the first type of droplets, the third width is greater than a particle size of each of the second type of droplets and the fourth width is less than the particle size of each of the second type of droplets.

In some embodiments, the microfluidic chip further comprises a sorting channel located upstream of the shunt structure, the sorting channel comprises a first branch and a second branch, the first branch communicates with the first channel and the second channel, and the second branch communicates with the third channel and the fourth channel.

In some embodiments, each shunt structure further comprises an auxiliary channel communicated with the second channel, the auxiliary channel is between the second channel and the first confluence, and the first channel and the auxiliary channel merge at the first confluence, the area of the first cross-section is greater than the area of the second cross-section, the auxiliary channel has a variable width in a fifth direction, the fifth direction is perpendicular to a flow direction of the second fluid in the auxiliary channel.

In some embodiments, the auxiliary channel comprises a first section and a second section which are alternately arranged, the first section has a fifth width in the fifth direction, the second section has a sixth width in the fifth direction, the fifth width is smaller than the sixth width.

In some embodiments, the at least one shunt structure is a plurality of shunt structures, and the plurality of shunt structures are arranged at intervals from each other.

In some embodiments, the microfluidic chip further comprises a droplet generation unit, the droplet generation unit is located upstream of the shunt structure and communicates with the shunt structure.

In some embodiments, the microfluidic chip further comprises a collection unit, the collection unit is located downstream of the shunt structure and communicates with the shunt structure.

According to another aspect of the present disclosure, a method of controlling a flow velocity of a fluid in a microfluidic chip is provided, the method comprising: providing the microfluidic chip described in any of the previous embodiments; making a flow velocity of the first channel greater than a flow velocity of the second channel by controlling at least one of a ratio of the area of the first cross-section to the area of the second cross-section and a ratio of the first length to the second length, to make the first fluid flow into the first channel and the second fluid flow into the second channel.

In some embodiments, the making a flow velocity of the first channel greater than a flow velocity of the second channel by controlling at least one of a ratio of the area of the first cross-section to the area of the second cross-section and a ratio of the first length to the second length, comprises: controlling the area of the first cross-section to be equal to the area of the second cross-section and the first length to be less than the second length, such that a relationship between a first ratio of a second flow velocity of the second channel to a first flow velocity of the first channel and a second ratio of the first length to the second length is substantially linear.

In some embodiments, the making a flow velocity of the first channel greater than a flow velocity of the second channel by controlling at least one of a ratio of the area of the first cross-section to the area of the second cross-section and a ratio of the first length to the second length, comprises: arranging a shape of the first cross-section to be circular and the first cross-section to have a first diameter, arranging a shape of the second cross-section to be circular and the second cross-section to have a second diameter, controlling the first length to be equal to the second length and the first diameter to be greater than the second diameter, such that a relationship between a first ratio of a second flow velocity of the second channel to a first flow velocity of the first channel and a third ratio of a square of the second diameter to a square of the first diameter is substantially linear.

According to yet another aspect of the present disclosure, a method of using a microfluidic chip is provided, the method comprising: providing the microfluidic chip described in any of the previous embodiments; predisposing an auxiliary stabilizer comprising at least one of an inorganic salt and a polyhydric alcohol in the second channel; generating liquid comprising droplets by the microfluidic chip, flowing the droplets in the liquid into the first channel, flowing a continuous phase fluid accompanying the droplets in the liquid into the second channel, a first flow velocity of the droplets in the first channel being greater than a second flow velocity of the continuous phase fluid in the second channel; dissolving the auxiliary stabilizer by the continuous phase fluid and flowing the auxiliary stabilizer carried by the continuous phase fluid along the second channel; and merging the continuous phase fluid dissolved the auxiliary stabilizer with the droplets at the first confluence of the microfluidic chip.

In some embodiments, each shunt structure further comprises a third channel, the first channel, the second channel and the third channel merge at the first confluence of the microfluidic chip, the third channel has a third cross-section and a third length, the third cross-section is perpendicular to a flow direction of the fluid in the third channel, the area of the first cross-section, the area of the second cross-section and an area of the third cross-section are equal, and the first length is less than the second length and the third length. The method comprises: predisposing a first auxiliary stabilizer and a second auxiliary stabilizer different from the first auxiliary stabilizer in the second channel and the third channel respectively, the first auxiliary stabilizer comprising at least one of an inorganic salt and a polyhydric alcohol, and the second auxiliary stabilizer comprising at least one of an inorganic salt and a polyhydric alcohol; generating the liquid by the microfluidic chip, flowing the droplets in the liquid into the first channel, and flowing the continuous phase fluid accompanying the droplets in the liquid into the second channel and the third channel respectively, a first flow velocity of the droplets in the first channel being greater than a second flow velocity of the continuous phase fluid in the second channel and a third flow velocity of the continuous phase fluid in the third channel; dissolving the first auxiliary stabilizer by the continuous phase fluid flowing into the second channel and flowing the first auxiliary stabilizer carried by the continuous phase fluid along the second channel, dissolving the second auxiliary stabilizer by the continuous phase fluid flowing into the third channel and flowing the second auxiliary stabilizer carried by the continuous phase fluid along the third channel; and merging the continuous phase fluid dissolved the first auxiliary stabilizer, the continuous phase fluid dissolved the second auxiliary stabilizer, and the droplets at the first confluence.

In some embodiments, each shunt structure further comprises a third channel, a fourth channel and a connecting channel, the third channel and the fourth channel merge at a second confluence of the microfluidic chip, the connecting channel communicates with the first confluence and the second confluence respectively, the third channel has a third cross-section, the fourth channel has a fourth cross-section, the third cross-section is perpendicular to a flow direction of the fluid in the third channel, the fourth cross-section is perpendicular to a flow direction of the fluid in the fourth channel, the area of the first cross-section is greater than the area of the second cross-section, and an area of the third cross-section is greater than an area of the fourth cross-section. The method comprises: predisposing a first auxiliary stabilizer and a second auxiliary stabilizer different from the first auxiliary stabilizer in the second channel and the fourth channel respectively, the first auxiliary stabilizer comprising at least one of an inorganic salt and a polyhydric alcohol, the second auxiliary stabilizer comprising at least one of an inorganic salt and a polyhydric alcohol; generating liquid by the microfluidic chip, the liquid comprising a first type of droplets, a second type of droplets and a continuous phase fluid accompanying the first type of droplets and the second type of droplets, the first type of droplets flowing into the first channel, the second type of droplets flowing into the third channel, the continuous phase fluid flowing into the second channel and the fourth channel respectively, a first flow velocity of the first type of droplets in the first channel being greater than a second flow velocity of the continuous phase fluid in the second channel, a third flow velocity of the second type of droplets in the third channel being greater than a fourth flow velocity of the continuous phase fluid in the fourth channel; dissolving the first auxiliary stabilizer by the continuous phase fluid flowing into the second channel and flowing the first auxiliary stabilizer carried by the continuous phase fluid along the second channel, and dissolving the second auxiliary stabilizer by the continuous phase fluid flowing into the fourth channel and flowing the second auxiliary stabilizer carried by the continuous phase fluid along the fourth channel; and merging the continuous phase fluid dissolved the first auxiliary stabilizer and the first type of droplets at the first confluence to form a first liquid, merging the continuous phase fluid dissolved the second auxiliary stabilizer and the second type of droplets at the second confluence to form a second liquid, and merging the first liquid and the second liquid via the connecting channel.

In some embodiments, the microfluidic chip further comprises a sorting channel located upstream of the shunt structure, the sorting channel comprises a first branch and a second branch, the first branch communicates with the first channel and the second channel, the second branch communicates with the third channel and the fourth channel. The generating liquid by the microfluidic chip comprises: detecting in real time the liquid generated by the microfluidic chip at the sorting channel by a detection device; in response to detecting the first type of droplets, flowing the first type of droplets and the continuous phase fluid accompanying the first type of droplets into the first branch of the sorting channel by applying an external force, flowing the first type of droplets into the first channel through the first branch, and flowing the continuous phase fluid accompanying the first type of droplets into the second channel through the first branch; in response to detecting the second type of droplets, flowing the second type of droplets and the continuous phase fluid accompanying the second type of droplets into the second branch of the sorting channel by applying an external force, flowing the second type of droplets into the third channel through the second branch, and flowing the continuous phase fluid accompanying the second type of droplets into the fourth channel through the second branch.

In some embodiments, each shunt structure further comprises an auxiliary channel communicated with the second channel, the auxiliary channel is between the second channel and the first confluence, the first channel and the auxiliary channel merge at the first confluence, the area of the first cross-section is greater than the area of the second cross-section, the auxiliary channel has a variable width in a fifth direction, the fifth direction is perpendicular to a flow direction of the continuous phase fluid in the auxiliary channel. The dissolving the auxiliary stabilizer by the continuous phase fluid and flowing the auxiliary stabilizer carried by the continuous phase fluid along the second channel, further comprises: dissolving the auxiliary stabilizer by the continuous phase fluid and flowing the auxiliary stabilizer carried by the continuous phase fluid along the second channel and the auxiliary channel, and changing the flow velocity of the continuous phase fluid carrying the auxiliary stabilizer in the auxiliary channel with the change of the width of the auxiliary channel.

In some embodiments, each of the droplets has a water-in-oil structure.

### BRIEF DESCRIPTION OF THE DRAWINGS

In order to more clearly describe the technical solutions in the embodiments of the present disclosure, the drawings that need to be used in the embodiments will be briefly described. Apparently, the drawings described in the following are only some embodiments of the present disclosure, and those skilled in the art can obtain other drawings according to these drawings without creative efforts.

FIG. 1 illustrates a schematic structural diagram of a microfluidic chip according to an embodiment of the present disclosure;

FIG. 2 illustrates an enlarged view of region I in FIG. 1;

FIG. 3 illustrates a flow velocity simulation diagram of the fluid in the shunt structure during movement;

FIG. 4 illustrates a particle trajectory simulation diagram of the fluid in the shunt structure during movement;

FIG. 5 illustrates a simulation diagram of the correlation between the flow velocity of the channel and the length of the channel when the diameters of the channels of the shunt structure are equal;

FIG. 6 illustrates a simulation diagram of the correlation between the flow velocity of the channel and the diameter of the channel when the lengths of the channels of the shunt structure are equal;

FIG. 7 illustrates another arrangement of shunt structures in a microfluidic chip according to an embodiment of the present disclosure;

FIG. 8 illustrates a schematic structural diagram of a microfluidic chip according to another embodiment of the present disclosure;

FIG. 9 illustrates a schematic structural diagram of a microfluidic chip according to yet another embodiment of the present disclosure;

FIG. 10 illustrates a schematic structural diagram of a microfluidic chip according to yet another embodiment of the present disclosure;

FIG. 11 illustrates a schematic structural diagram of a microfluidic chip according to yet another embodiment of the present disclosure;

FIG. 12 illustrates a flowchart of a method of controlling the flow velocity of a fluid in a microfluidic chip according to an embodiment of the present disclosure; and

FIG. 13 illustrates a flowchart of a method of using a microfluidic chip according to an embodiment of the present disclosure.

### DETAILED DESCRIPTION OF THE DISCLOSURE

The following will clearly and completely describe the technical solutions in the embodiments of the present disclosure with reference to the drawings in the embodiments of the present disclosure. Apparently, the described embodiments are only some, but not all, of the embodiments of the present disclosure. Based on the embodiments in the present disclosure, all other embodiments obtained by persons of ordinary skill in the art without making creative efforts belong to the protection scope of the present disclosure.

Droplet microfluidic technology refers to the following technology: the interaction between two-phase fluids that are immiscible with each other is used to disperse one of the two-phase fluids (such as the dispersed-phase fluid) into independent micro-droplets in the micro-channel, and the flow pattern and length of the micro-droplets are precisely controlled. Micro-droplets have important applications in the fields of in vitro diagnosis, controlled drug release, virus detection, synthesis of granular materials, and catalysts. For example, in the field of in vitro diagnostics, gene chips, protein chips, digital polymerase chain reaction, etc. all need to use micro-droplets.

The inventors of the present application found that during the generation of droplets by using the microfluidic chip and the subsequent movement of the droplets in the microfluidic chip, the environment in the microfluidic chip will impact on the stability of the droplets. As a result, it is easy for the droplets to break or to merge with each other, so that the droplets lack stability. When such broken or merged droplets are used for biochemical detection, the accuracy of the experimental data cannot be guaranteed, thereby failing to provide strong data support for subsequent diagnostic detection.

In order to improve the stability of droplets, the inventors of the present application provide various microfluidic chips.

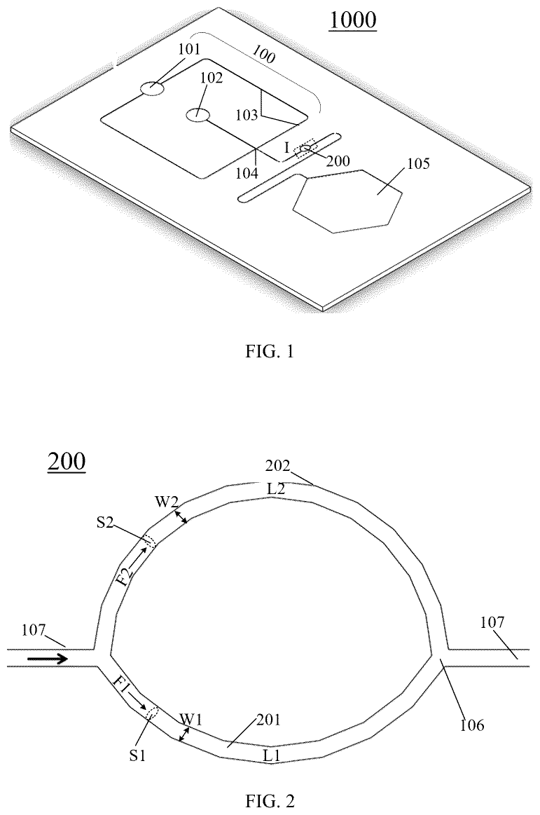

FIG. 1 illustrates a microfluidic chip 1000 as an embodiment, and the microfluidic chip 1000 comprises at least one shunt structure 200. As an example, FIG. 1 illustrates only one shunt structure 200. FIG. 2 illustrates an enlarged view of region I in FIG. 1. Referring to FIG. 1 and FIG. 2, the shunt structure 200 comprises at least two channels, the at least two channels comprise a first channel 201 and a second channel 202, the first channel 201 is configured to allow the first fluid to flow therein, the second channel 202 is configured to allow the second fluid to flow therein, and the first fluid and the second fluid merge at a first confluence 106 of the microfluidic chip 1000. The first channel 201 has a first cross-section S1 and a first length L1, the second channel 202 has a second cross-section S2 and a second length L2. The first cross-section Si is perpendicular to the flow direction F1 of the first fluid in the first channel 201, and the second cross-section S2 is perpendicular to the flow direction F2 of the second fluid in the second channel 202. The area of the first cross-section S1 is greater than or equal to the area of the second cross-section S2, and the first length L1 is less than or equal to the second length L2.

By making the area of the first cross-section S1 greater than or equal to the area of the second cross-section S2 and the first length L1 less than or equal to the second length L2, the flow velocity of the first channel 201 can be greater than the flow velocity of the second channel 202, the first fluid flows into the first channel 201 with a greater flow velocity, and the second fluid flows into the second channel 202 with a lower flow velocity, so that the first fluid is separated from the second fluid.

The area of the first cross-section S1 is greater than or equal to the area of the second cross-section S2 and the first length L1 is less than or equal to the second length L2, which may comprise the following cases: the area of the first cross-section S1 is greater than the area of the second cross-section S2, and the first length L1 is less than the second length L2; the area of the first cross-section S1 is greater than the area of the second cross-section S2, and the first length L1 is equal to the second length L2; the area of the first cross-section S1 is equal to the area of the second cross-sections S2, and the first length L1 is less than the second length L2; and the area of the first cross-section S1 is equal to the area of the second cross-section S2, and the first length L1 is equal to the second length L2. In any case, according to the relationship between the length and/or width of the channel and the flow velocity, the flow velocity of the first channel 201 is greater than the flow velocity of the second channel 202. For example, in the special case where the area of the first cross-section S1 is equal to the area of the second cross-section S2 and the first length L1 is equal to the second length L2, although the area of the first cross-section S1 is equal to the area of the second cross-section S2, by making the width of the first cross-section S1 along a first direction larger than the width of the second cross-section S2 along a second direction, the flow velocity of the first channel 201 is still greater than that of the second channel 202.

As illustrated in FIG. 1 and FIG. 2, the microfluidic chip 1000 may further comprise a droplet generation unit 100 and a collection unit 105. The droplet generation unit 100 is located upstream of the shunt structure 200 and communicates with the shunt structure 200, and the collection unit 105 is located downstream of the shunt structure 200 and communicates with the shunt structure 200. The droplet generation unit 100 comprises: a first container 101, which is configured to accommodate a continuous phase (such as an oil phase) fluid, in which a surfactant may be mixed; a second container 102, which is configured to accommodate a dispersed phase (such as an aqueous phase) fluid, in which, for example, a cell suspension may be comprised; and a delivery channel 103, which communicates with the first container 101 and the second container 102 respectively. The continuous phase fluid and the dispersed phase fluid merge at the intersection 104 of the delivery channel 103, and under the extrusion of the delivery channel 103, the continuous phase fluid and the dispersed phase fluid generate droplets with a water-in-oil structure and some continuous phase fluid accompanying the droplets at the intersection 104. The droplet comprises a single cell. The surfactant in the continuous phase fluid acts as an emulsifier, on the one hand the surfactant has good biocompatibility, on the other hand the surfactant can provide good stability for the generated droplets. These droplets and the continuous phase fluid accompanying the droplets continue to flow along the main channel 107, and the particle size of the droplet is generally larger than that of a single particle (e.g., a single oil phase particle) in the continuous phase fluid. Since the flow velocity of the first channel 201 of the shunt structure 200 is greater than the flow velocity of the second channel 202 of the shunt structure 200, the droplets with larger particle sizes will flow into the first channel 201 of the shunt structure 200, and the continuous phase fluids with smaller particle sizes will flow into the second channel 202 of the shunt structure 200. In this case, the first fluid may refer to the droplets, and the second fluid may refer to the continuous phase fluid mixed with the surfactant. The droplets and the continuous phase fluid merge at the first confluence 106 and continue to flow along the main channel 107 and then flow into the collection unit 105.

In an embodiment, as illustrated in FIG. 2, the area of the first cross-section S1 of the first channel 201 of the shunt structure 200 is larger than the area of the second cross-section S2 of the second channel 202 of the shunt structure 200, and, the first length L1 of the first channel 201 is smaller than the second length L2 of the second channel 202. The first channel 201 and the second channel 202 merge at the first confluence 106. In an example, the first cross-section 51 of the first channel 201 has a first width W1 in a first direction, which is perpendicular to the flow direction of the first fluid (such as the droplets) in the first channel 201; the second cross-section S2 of the second channel 202 has a second width W2 in a second direction, which is perpendicular to the flow direction of the second fluid (such as the continuous phase fluid mixed with the emulsifier) in the second channel 202. In an example, the first width W1 is larger than the particle size of the droplet, and the second width W2 is smaller than the particle size of the droplet. In other words, the first channel 201 is wider and shorter than the second channel 202, its width is larger than the particle size of the generated droplets and allows the droplets to pass through, and has a greater flow velocity; while the second channel 202 is narrower and longer than the first channel 201, its width is smaller than the particle size of the generated droplets and does not allow the droplets to pass through, and has a lower flow velocity. With such design, the droplets flow into the first channel 201 with a greater flow velocity and avoid flowing into the second channel 202, while the continuous phase fluid flows into the second channel 202 with a lower flow velocity.

In some embodiments, before generating droplets by the microfluidic chip 1000, an auxiliary stabilizer can be predisposed in the second channel 202 of the shunt structure 200, and the auxiliary stabilizer may comprise at least one of polyhydric alcohol and inorganic salt. Polyhydric alcohol and/or inorganic salt can be used as auxiliary emulsifier to improve the stability of droplets. As mentioned above, the continuous phase (such as the oil phase) fluid is mixed with the emulsifier, and the droplets generated by mixing the continuous phase fluid and the dispersed phase fluid have a water-in-oil structure, the outside of the droplet is an oil phase mixed with the emulsifier, the inside of the droplet is the water phase. When the droplets merge with the continuous phase fluid flowing into the second channel 202 and dissolved with polyhydric alcohol and/or inorganic salt at the first confluence 106, the polyhydric alcohol can reduce the solubility of the emulsifier of the droplets in water, make the emulsifier stay in the oil phase more, so that the lipophilicity of the emulsifier increases and the hydrophilicity of the emulsifier decreases, and the emulsifying ability is strengthened. The “salting-out” effect of inorganic salts can compete with the emulsifier in the droplets for water molecules, thus helping to reduce the solubility of the emulsifier in water. The emulsifier is an amphiphilic substance with a positive charge at the lipophilic end and a negative charge at the hydrophilic end. Therefore, the surface of the droplet with the water-in-oil structure is positively charged, and will absorb negative ions of inorganic salts to form a diffuse electric double layers. Since each droplet has positive and negative electric double layers, they repel each other and are not easy to merge with each other. According to specific circumstances, only polyhydric alcohols, only inorganic salts, or both polyhydric alcohols and inorganic salts may be predisposed in the second channel 202. The polyhydric alcohol may be a variety of suitable materials comprising, but not limited to, ethanol, n-butanol, isobutanol, n-pentanol, sorbitol, glycerin. The inorganic salt may be any suitable material comprising, but not limited to, sodium chloride, magnesium sulfate, calcium chloride.

After generating droplets by the microfluidic chip 1000, these droplets and the continuous phase fluid accompanying the droplets flow along the main channel 107. The first channel 201 of the shunt structure 200 has the first width W1 larger than the particle size of the droplet and a shorter length L1, thus has a larger flow velocity, so that the droplets flow into the first channel 201. Therefore, the droplets flowing into the first channel 201 can avoid contact with the polyhydric alcohol and/or inorganic salt predisposed in the second channel 202. In this way, the negative impact on the charge and structure of the droplet due to the sudden increase of the fluid concentration caused by the presence of the polyhydric alcohol and/or inorganic salt can be avoided, thereby avoiding the breakage of the droplets and improving the stability of the droplets. The continuous phase fluid flows along the main channel 107 into the second channel 202 with the second width W2 smaller than the particle size of the droplet and a longer length L2. Since the second channel 202 has a relatively lower flow velocity, the continuous phase fluid can fully contact and dissolve the polyhydric alcohol and/or inorganic salt predisposed in the second channel 202 during flowing through the second channel 202, and carry the polyhydric alcohol and/or inorganic salt to flow forward along the second channel 202. The droplets in the first channel 201 and the continuous phase fluid dissolved with the polyhydric alcohol and/or inorganic salt in the second channel 202 merge at the first confluence 106. As mentioned above, the polyhydric alcohol and/or inorganic salt can reduce the solubility of emulsifier in water of droplets with a water-in-oil structure, increase the lipophilicity of the emulsifier and reduce the hydrophilicity of the emulsifier, and enhance the emulsifying ability of the emulsifier. Therefore, by predisposing polyhydric alcohol and/or inorganic salt in the second channel 202, the microenvironment of the channel after the droplet is generated can be changed to prevent the droplets from breaking or merging, thereby improving the stability of the droplets.

It should be noted that, as used herein, the term “particle size of a droplet” refers to the size of a droplet, that is, the length of a droplet in a certain direction. For example, when the shape of the droplet is spherical, the term “particle size of a droplet” refers to the diameter of the droplet. When the shape of the droplet is rod, the term “particle size of a droplet” refers to the length of the droplet in the direction of the shorter side.

In some embodiments, as illustrated in FIG. 2, the shape of the first channel 201 and the second channel 202 of the shunt structure 200 is arc, such as a circular arc. The design of the arc-shaped channel can make the fluid flow more smoothly in the channel and avoid the “dead volume” of fluid in the channel. The first channel 201 and the second channel 202 of the shunt structure 200 are formed as asymmetrical double arc channels, the first channel 201 is wide and short, and the second channel 202 is narrow and long.

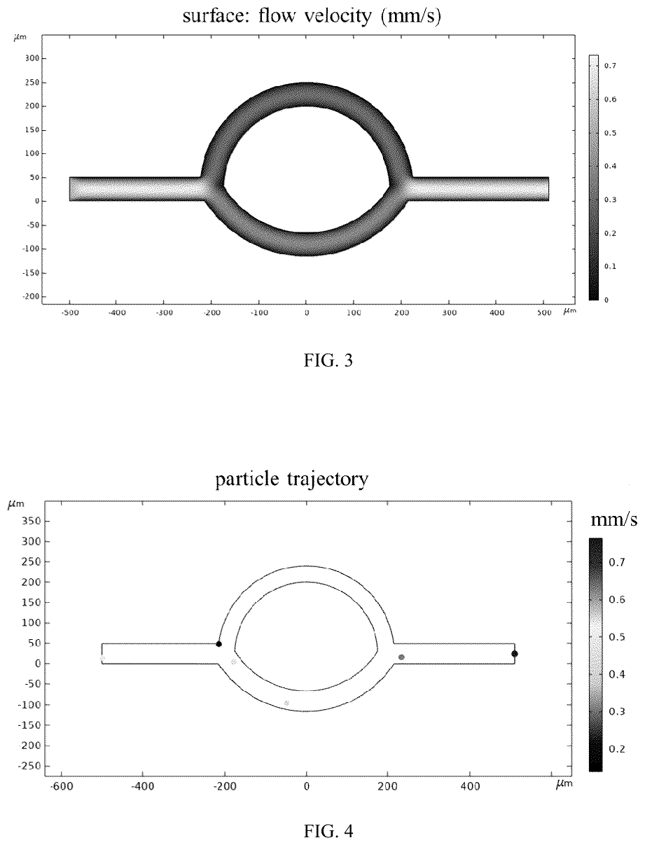

FIG. 3 is a simulation diagram of the flow velocity of the fluid in the shunt structure 200 during the movement. The flow velocity of the first channel 201 is greater than that of the second channel 202, which indicates that the fluid can be divided at the shunt structure 200, and the flow velocity is different. Compared with the first channel 201, the second channel 202 has a lower flow velocity, which is beneficial for the continuous phase fluid to fully contact and dissolve the polyhydric alcohol and/or inorganic salt predisposed in the second channel 202.

FIG. 4 is a simulation diagram of particle trajectory of fluid in the shunt structure 200 during movement. As illustrated in the figure, the particles only exist in the first channel 201 of the shunt structure 200 and the main channel 107, but not in the second channel 202 of the shunt structure 200. This indicates that the droplets flow into the first channel 201 with a relatively greater flow velocity, and move in the first channel 201. As mentioned above, the droplets and the continuous phase fluid dissolved with the polyhydric alcohol and/or inorganic salt merge at the first confluence 106, and the polyhydric alcohol and/or inorganic salt in the system act as co-emulsifiers, compete with the emulsifier in the droplets for more water molecules, reduce the solubility of the emulsifier in water, increase the lipophilicity of the emulsifier and decrease the hydrophilicity of the emulsifier, and improve the emulsifying ability of the emulsifier, thereby increasing the stability of the droplets.

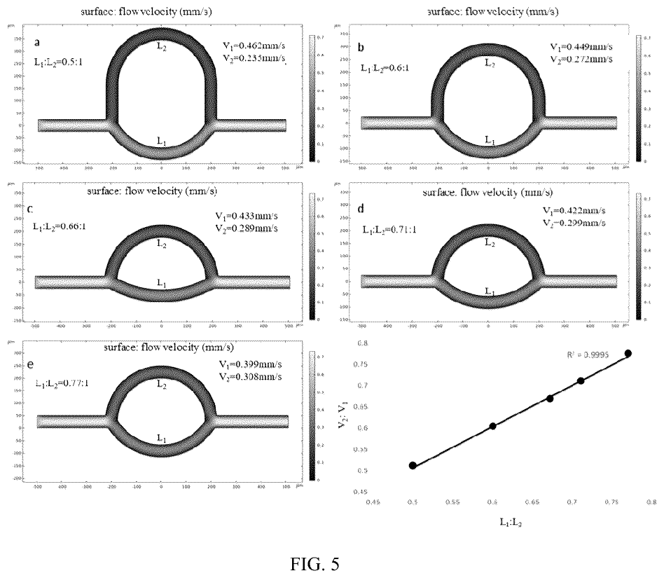

FIG. 5 is a simulation diagram of the correlation between the flow velocity of the channel and the length of the channel when the area of the first cross-section S1 of the first channel 201 of the shunt structure 200 is equal to the area of the second cross-section S2 of the second channel 202. FIG. 5 lists the flow velocities corresponding to five different channel length ratios. As illustrated in FIG. 5(a), the ratio of the first length L1 of the first channel 201 to the second length L2 of the second channel 202 of the shunt structure 200 is L1:L2=0.5:1, in this case, the first flow velocity V1 of the first channel 201 is 0.462 mm/s, and the second flow velocity V2 of the second channel 202 is 0.235 mm/s. As illustrated in FIG. 5(b), the ratio of the first length L1 of the first channel 201 to the second length L2 of the second channel 202 of the shunt structure 200 is L1:L2=0.6:1, in this case, the first flow velocity V1 of the first channel 201 is 0.449 mm/s, and the second flow velocity V2 of the second channel 202 is 0.272 mm/s. As illustrated in FIG. 5(c), the ratio of the first length L1 of the first channel 201 to the second length L2 of the second channel 202 of the shunt structure 200 is L1:L2=0.66:1, in this case, the first flow velocity V1 of the first channel 201 is 0.433 mm/s, and the second flow velocity V2 of the second channel 202 is 0.289 mm/s. As illustrated in FIG. 5(d), the ratio of the first length L1 of the first channel 201 to the second length L2 of the second channel 202 of the shunt structure 200 is L1:L2=0.71:1, in this case, the first flow velocity V1 of the first channel 201 is 0.422 mm/s, and the second flow velocity V2 of the second channel 202 is 0.299 mm/s. As illustrated in FIG. 5(e), the ratio of the first length L1 of the first channel 201 to the second length L2 of the second channel 202 of the shunt structure 200 is L1:L2=0.77:1, in this case, the first flow velocity V1 of the first channel 201 is 0.399 mm/s, and the second flow velocity V2 of the second channel 202 is 0.308 mm/s. It can be seen that the shorter first channel 201 always has a greater flow velocity, and the flow velocity is inversely proportional to the length of the channel. In the five sets of data shown in (a)-(e), the smaller the ratio of L1:L2, the larger V1 and the smaller V2 (such as FIG. 5(a)); the larger the ratio of L1:L2, the smaller V1 and the larger V2 (such as FIG. 5(e)). By performing linear fitting on the five sets of data illustrated in (a)-(e), it can be concluded that, the relationship between the first ratio of the second flow velocity V2 in the second channel 202 to the first flow velocity V1 in the first channel 201 and the second ratio of the first length L1 to the second length L2 is substantially linear. The lower right of FIG. 5 illustrates the fitted linear relationship diagram, the square value R² (also called the coefficient of determination) of the linear correlation coefficient between the first ratio and the second ratio is 0.9995, which shows that a very good correlation exists between the first ratio and the second ratio. Therefore, it can be seen from FIG. 5 that the flow velocity of the first channel 201 and the second channel 202 can be adjusted by changing the ratio of lengths of the first channel 201 and the second channel 202 of the shunt structure 200. The shorter first channel 201 always has a greater flow velocity, so the droplets flow into the shorter first channel 201 with a greater flow velocity.

It should be noted that, FIG. 5 describes the relationship between the length of the channel and the flow velocity by taking the shunt structure 200 as an example, but this does not mean that this relationship is only applicable to the shunt structure 200. In fact, the relationship between the length of the channel and the flow velocity described above is applicable to all shunt structures described in the various embodiments of the present disclosure (comprising the shunt structures 300, 400, 500, 600 etc. to be described below).

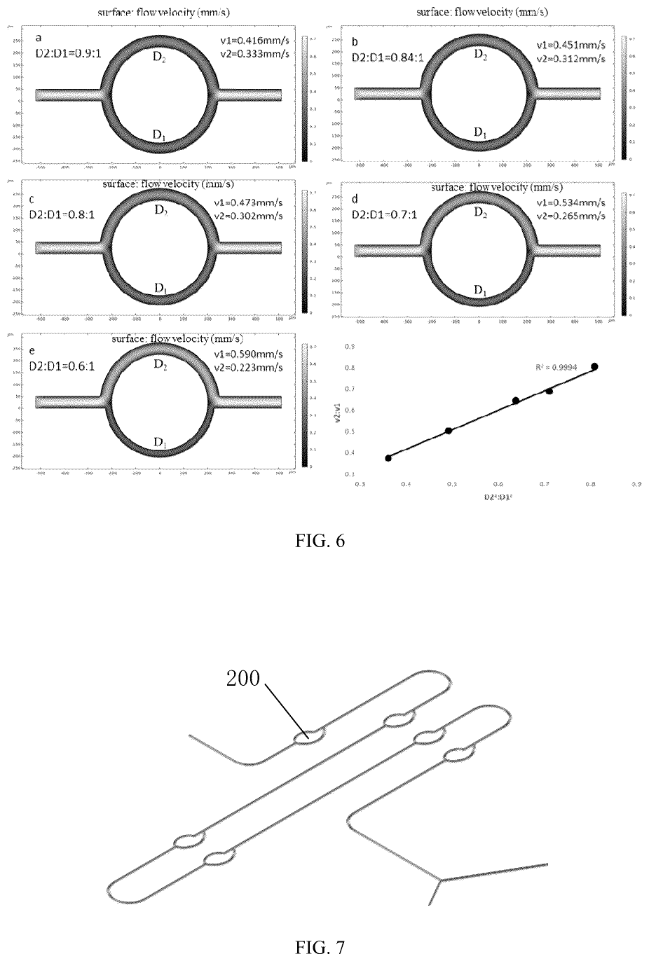

FIG. 6 is a simulation diagram of the correlation between the flow velocity of the channel and the diameter of the cross-section of the channel when the first length L1 of the first channel 201 of the shunt structure 200 is equal to the second length L2 of the second channel 202. The first cross-section S1 of the first channel 201 has a circular shape and has a first diameter D1, the second cross-section S2 of the second channel 202 has a circular shape and has a second diameter D2, and the first diameter D1 is larger than the second diameter D2. FIG. 6 lists the flow velocities corresponding to five different diameter ratios. As illustrated in FIG. 6(a), the ratio of the second diameter D2 of the second channel 202 of the shunt structure 200 to the first diameter D1 of the first channel 201 is D2:D1=0.9:1, in this case, the first flow velocity V1 of the first channel 201 is 0.416 mm/s, and the second flow velocity V2 of the second channel 202 is 0.333 mm/s. As illustrated in FIG. 6(b), the ratio of the second diameter D2 of the second channel 202 of the shunt structure 200 to the first diameter D1 of the first channel 201 is D2:D1=0.84:1, in this case, the first flow velocity V1 of the first channel 201 is 0.451 mm/s, and the second flow velocity V2 of the second channel 202 is 0.312 mm/s. As illustrated in FIG. 6(c), the ratio of the second diameter D2 of the second channel 202 of the shunt structure 200 to the first diameter D1 of the first channel 201 is D2:D1=0.8:1, in this case, the first flow velocity V1 of the first channel 201 is 0.473 mm/s, and the second flow velocity V2 of the second channel 202 is 0.302 mm/s. As illustrated in FIG. 6(d), the ratio of the second diameter D2 of the second channel 202 of the shunt structure 200 to the first diameter D1 of the first channel 201 is D2:D1=0.7:1, in this case, the first flow velocity V1 of the first channel 201 is 0.534 mm/s, and the second flow velocity V2 of the second channel 202 is 0.265 mm/s. As illustrated in FIG. 6(e), the ratio of the second diameter D2 of the second channel 202 of the shunt structure 200 to the first diameter D1 of the first channel 201 is D2:D1=0.6:1, in this case, the first flow velocity V1 of the first channel 201 is 0.590 mm/s, and the second flow velocity V2 of the second channel 202 is 0.223 mm/s. It can be seen that the channel with larger diameter always has a higher flow velocity. In the five sets of data illustrated in (a)-(e), the smaller the ratio of D2:D1, the larger V1 and the smaller V2 (such as FIG. 6(e)); the larger the ratio of D2:D1, the smaller V1 and the larger V2 (such as FIG. 6(a)). By performing linear fitting on the five sets of data shown in (a)-(e), it can be seen that the relationship between the first ratio of the second flow velocity V2 in the second channel 202 to the first flow velocity V1 in the first channel 201 and the third ratio of the square of the second diameter D2 to the square of the first diameter D1 is substantially linear. The lower right of FIG. 6 illustrates the fitted linear relationship diagram, the square value R² (also called the coefficient of determination) of the linear correlation coefficient between the first ratio and the third ratio is 0.9994, which shows that a very good correlation exists between the first ratio and the third ratio. Therefore, it can be seen from FIG. 6 that the flow velocities of the first channel 201 and the second channel 202 can be adjusted by changing the ratio of diameters of the first channel 201 and the second channel 202 of the shunt structure 200. The first channel 201 with a larger diameter always has a greater flow velocity, so the droplets flow into the first channel 201 with a larger diameter in a greater flow velocity.

It should be noted that FIG. 6 uses the shunt structure 200 as an example to describe the relationship between the diameter of channel and the flow velocity, but this does not mean that this relationship is only applicable to the shunt structure 200. In fact, the relationship between the diameter of channel and the flow velocity described above is applicable to all shunt structures described in the various embodiments of the present disclosure (comprising the shunt structures 300, 400, 500, 600 etc. to be described below).

FIG. 7 illustrates another arrangement of the shunt structure 200 in the microfluidic chip 1000. As mentioned above, the microfluidic chip 1000 comprises at least one shunt structure 200, and FIG. 7 illustrates a plurality of shunt structures 200, which are disposed in the main channel 107 of the microfluidic chip 1000 at an interval from each other. The number of shunt structures 200 depends on the concentration of the polyhydric alcohol and/or inorganic salt, and the embodiment of the present disclosure does not specifically limit the number of shunt structures 200.

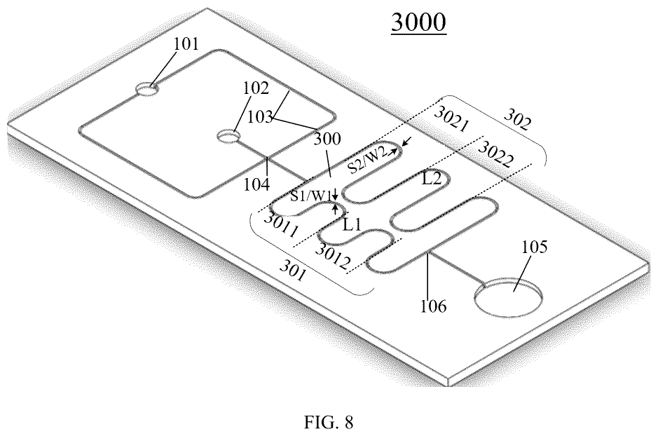

FIG. 8 illustrates a schematic structural diagram of a microfluidic chip 3000. The microfluidic chip 3000 illustrated in FIG. 8 has basically the same structure as the microfluidic chip 1000 illustrated in FIG. 1 except that the shunt structure 300 is different, and therefore the same reference numerals are used to refer to the same components. For example, the microfluidic chip 3000 also comprises structures such as the first container 101, the second container 102, the delivery channel 103 (comprising the intersection 104), and the collection unit 105. Therefore, for the detailed structures and functions of components in FIG. 8 that have the same reference numerals as in FIG. 1, reference may be made to the description of FIG. 1, and details are not repeated here. For brevity, only the differences are described below.

As illustrated in FIG. 8, the shunt structure 300 comprises a first channel 301 and a second channel 302. The area of the first cross-section S1 of the first channel 301 is equal to the area of the second cross-section S2 of the second channel 302, but the first length L1 of the first channel 301 is smaller than the second length L2 of the second channel 302, and the first channel 301 and the second channel 302 merge at the first confluence 106. According to the correlation between the flow velocity and the length of the channel, the flow velocity of the first channel 301 is greater than that of the second channel 302, so the droplets pass through the first channel 301, and the auxiliary stabilizer (the polyhydric alcohol and/or inorganic salt) can be predisposed in the second channel 302.

The first cross-section S1 of the first channel 301 has a first width W1 in a first direction, which is perpendicular to the flow direction of the first fluid (such as the droplets) in the first channel 301; the second cross-section S2 of the second channel 302 has a second width W2 in a second direction, which is perpendicular to the flow direction of the second fluid (such as a continuous phase fluid mixed with an emulsifier) in the second channel 302. In an example, the first width W1 is equal to the second width W2 and both are greater than the particle size of the droplet.

As illustrated in FIG. 8, the first channel 301 of the shunt structure 300 comprises at least one section, two sections 3011 and 3012 are illustrated in the figure, the two sections 3011 and 3012 communicate with each other, and the shape of each section is S-shaped. The second channel 302 of the shunt structure 300 comprises at least one section, two sections 3021 and 3022 are illustrated in the figure, the two sections 3021 and 3022 communicate with each other, and the shape of each section is reverse S-shaped. The reverse S-shaped refers to the mirror symmetry of the “S” shape, that is, basically the shape of “ [symbol] ”. As illustrated in the figure, the number of sections of the first channel 301 is equal to the number of sections of the second channel 302, and each section of the first channel 301 is arranged correspondingly to a corresponding section of the second channel 302, for example, the section 3011 is arranged correspondingly to the section 3021, and the section 3012 is arranged correspondingly to the section 3022. The section 3011 has the same length as the section 3012, and the section 3021 has the same length as the section 3022, but the section 3021 has a length greater than the section 3011.

In addition to having all the technical effects of the shunt structure 200, the shunt structure 300 increases the length of the first channel 301 and the second channel 302 in a limited space by designing the first channel 301 and the second channel 302 into a bent S shape. Therefore, the dispersed phase fluid can have a longer contact with the polyhydric alcohol and/or inorganic salt predisposed in the second channel 302, thus the polyhydric alcohol and/or inorganic salt can be dissolved more fully in the continuous phase fluid. At the same time, since the length of the channel is increased in a limited space, the shunt structure 300 can help reduce the volume of the microfluidic chip 3000 compared with the conventional straight channel on the premise that the channel of the same length is required.

It should be noted that FIG. 8 only illustrates a possible shape of the first channel 301 and the second channel 302 as an example, but this does not limit that the first channel 301 and the second channel 302 are arranged to such shape. For example, in an alternative embodiment, the second channel 302 may have a greater number of sections, and the first channel 301 may have a smaller number of sections, so that the length of the second channel 302 is greater than that of the first channel 301. For those skilled in the art, all other technical solutions that can increase the lengths of the first channel 301 and the second channel 302 in a limited space obtained based on the embodiment of FIG. 8 should fall within the protection scope of the present disclosure.

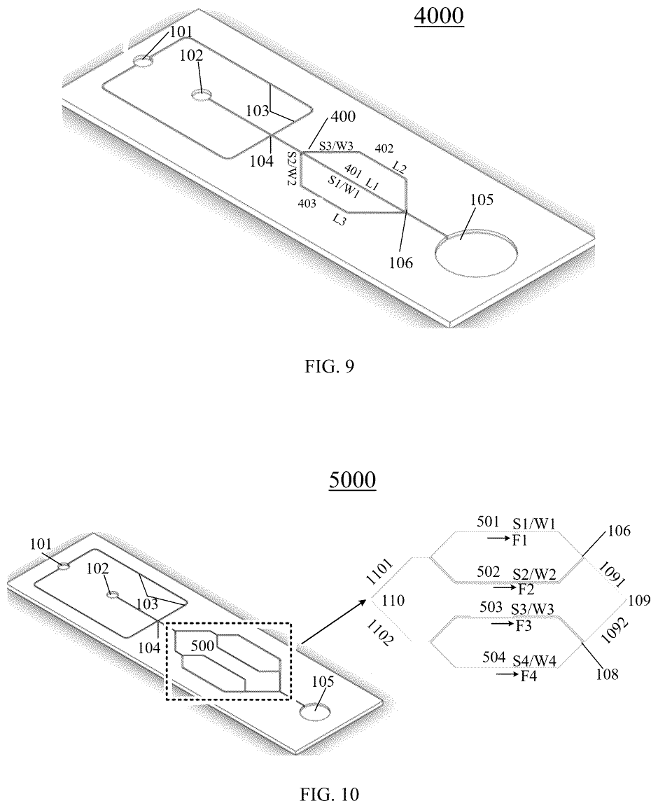

FIG. 9 illustrates a schematic structural diagram of a microfluidic chip 4000. The microfluidic chip 4000 illustrated in FIG. 9 has basically the same structure as the microfluidic chip 1000 illustrated in FIG. 1 except that the shunt structure 400 is different, and therefore the same reference numerals are used to refer to the same components. For example, the microfluidic chip 4000 also comprises structures such as the first container 101, the second container 102, the delivery channel 103 (comprising the intersection 104), and the collection unit 105. Therefore, the detailed structures and functions of components in FIG. 9 which have the same reference numerals as in FIG. 1, reference may be made to the description of FIG. 1, and details will not be repeated here. For brevity, only the differences are described below.

As illustrated in FIG. 9, the shunt structure 400 comprises a first channel 401, a second channel 402 and a third channel 403, the third channel 403 is configured to allow the second fluid to flow therein, and the second fluid is a continuous phase fluid. The first channel 401, the second channel 402 and the third channel 403 merge at the first confluence 106. The first channel 401 has a first cross-section S1 and a first length L1, the second channel 402 has a second cross-section S2 and a second length L2, and the third channel 403 has a third cross-section S3 and a third length L3. The first cross-section S1 is perpendicular to the flow direction of the first fluid (the droplets) in the first channel 401, the second cross-section S2 is perpendicular to the flow direction of the second fluid (the continuous phase fluid) in the second channel 402, and the third cross-section S3 is perpendicular to the flow direction of the second fluid (the continuous phase fluid) in the third channel 403. The areas of the first cross-section S1, the second cross-section S2, and the third cross-section S3 are equal, but the first length L1 is smaller than the second length L2 and the third length L3. According to the correlation between the flow velocity and the length of the channel, the flow velocity of the first channel 401 is greater than the flow velocity of the second channel 402 and the third channel 403. Therefore, the droplets pass through the first channel 401, and the first auxiliary stabilizer (the polyhydric alcohol and/or inorganic salt) and the second auxiliary stabilizer (the polyhydric alcohol and/or inorganic salt) different from the first auxiliary stabilizer can be respectively predisposed in the second channel 402 and the third channel 403. The first channel 401 is located between the second channel 402 and the third channel 403, and the second channel 402 and the third channel 403 are axisymmetric with respect to the first channel 401.

The first cross-section S1 of the first channel 401 has a first width W1 in a first direction, which is perpendicular to the flow direction of the first fluid (the droplets) in the first channel 401; the second cross-section S2 of the second channel 402 has a second width W2 in a second direction, which is perpendicular to the flow direction of the second fluid (the continuous phase fluid) in the second channel 402; the third cross-section S3 of the third channel 403 has a third width W3 in a third direction, which is perpendicular to the flow direction of the second fluid (the continuous phase fluid) in the third channel 403. In an example, the first width W1, the second width W2 and the third width W3 are equal and greater than the particle size of the droplet.

The droplets are generated by using the microfluidic chip 4000, and these droplets and the continuous phase fluid accompanying the droplets continue to flow along the main channel 107. According to the correlation between the flow velocity and the length of the channel, the flow velocity of the first channel 401 is greater than the flow velocity of the second channel 402 and the third channel 403, so the droplets flow into the first channel 401, and the continuous phase fluid flows into the second channel 402 and the third channel 403 respectively. The continuous phase fluid flowing into the second channel 402 can fully contact and dissolve the first auxiliary stabilizer predisposed in the second channel 402, and carry the first auxiliary stabilizer to flow along the second channel 402. The continuous phase fluid flowing into the third channel 403 can fully contact and dissolve the second auxiliary stabilizer predisposed in the third channel 403, and carry the second auxiliary stabilizer to flow along the third channel 403. The droplets in the first channel 401, the continuous phase fluid dissolved with the first auxiliary stabilizer in the second channel 402, and the continuous phase fluid dissolved with the second auxiliary stabilizer in the third channel 403 merge at the first confluence 106

In addition to having all the technical effects of the shunt structure 200, the shunt structure 400 adds a channel where another auxiliary stabilizer can be placed by additionally arranging the third channel 403. In this way, the first auxiliary stabilizer and the second auxiliary stabilizer can be respectively disposed in different channels, which can realize independent storage of different types of auxiliary stabilizers and avoid mutual interference.

FIG. 10 illustrates a schematic structural diagram of a microfluidic chip 5000. Except for the shunt structure 500 and the sorting channel 110, the microfluidic chip 5000 illustrated in FIG. 10 has basically the same structure as the microfluidic chip 1000 illustrated in FIG. 1, and therefore the same reference numerals are used to refer to the same components. For example, the microfluidic chip 5000 also comprises structures such as the first container 101, the second container 102, the delivery channel 103 (comprising the intersection 104), and the collection unit 105. Therefore, for the detailed structures and functions of components in FIG. 10 which have the same reference numerals as those in FIG. 1, reference may be made to the description of FIG. 1, and details will not be repeated here. For brevity, only the differences are described below.

The microfluidic chip 5000 also comprises a sorting channel 110, and the upper right of FIG. 10 is an enlarged view of the sorting channel 110 and the shunt structure 500. As illustrated, the sorting channel 110 is located between the intersection 104 and the shunt structure 500. The sorting channel 110 comprises a first branch 1101 and a second branch 1102. In some embodiments, the first branch 1101 and the second branch 1102 have the same length and cross-sectional area. The microfluidic chip 5000 generates droplets at the intersection 104. In some embodiments, the types of objects (such as cells, nucleic acid molecules, etc.) encapsulated by the droplets may be different, and thus the droplets may have different properties. Droplets with different properties are expected to be collected separately to facilitate subsequent experimental detection. The introduction of the sorting channel 110 can help sort these droplets with different properties. In some embodiments, the process of sorting the droplets with different properties may be roughly as follows: detecting the droplets generated by the microfluidic chip 5000 at the sorting channel 110 in real time by a detection device (such as an optical detection device); in response to detecting a first type of droplets having the first property, making the first type of droplets and a portion of the continuous phase fluid accompanying the first type of droplets flow into the first branch 1101 of the sorting channel 110 by applying an external force; in response to detecting a second type of droplets having a second property different from the first property, making the second type of droplets and another portion of the continuous phase fluid accompanying the second type of droplets flow into the second branch 1102 of the sorting channel 110 by applying an external force. The “external force” here may be various appropriate external forces, comprising but not limited to gas driving force, dielectric force generated by applying voltage to electrodes, etc., as long as the force ensures that the droplets with different properties can enter different branches of the sorting channel 110 under the action of the force.

The shunt structure 500 comprises a first channel 501, a second channel 502, a third channel 503, a fourth channel 504 and a connecting channel 109. The first branch 1101 of the sorting channel 110 communicates with the first channel 501 and the second channel 502, and the second branch 1102 of the sorting channel 110 communicates with the third channel 503 and the fourth channel 504. The first channel 501 and the second channel 502 merge at the first confluence 106, and the third channel 503 and the fourth channel 504 merge at the second confluence 108. The connecting channel 109 comprises a first sub-channel 1091 and a second sub-channel 1092, the first sub-channel 1091 communicates with the first confluence 106, and the second sub-channel 1092 communicates with the second confluence 108.

The first channel 501 has a first cross-section S1, the second channel 502 has a second cross-section S2, the third channel 503 has a third cross-section S3, and the fourth channel 504 has a fourth cross-section S4. The area of the first cross-section S1 is greater than the area of the second cross-section S2. According to the correlation between the flow velocity and the width of the channel, the flow velocity of the first channel 501 is greater than the flow velocity of the second channel 502. Therefore, the first type of droplets in the first branch 1101 flow into the first channel 501, and the continuous phase fluid in the first branch 1101 flows into the second channel 502. A first auxiliary stabilizer suitable for the first type of droplets may be predisposed in the second channel 502. Similarly, the area of the third cross-section S3 is greater than the area of the fourth cross-section S4, according to the correlation between the flow velocity and the width of the channel, the flow velocity of the third channel 503 is greater than the flow velocity of the fourth channel 504. Therefore, the second type of droplets in the second branch 1102 flow into the third channel 503, and the continuous phase fluid in the second branch 1102 flows into the fourth channel 504. A second auxiliary stabilizer suitable for the second type of droplets may be predisposed in the fourth channel 504. The continuous phase fluid flowing into the second channel 502 can fully contact and dissolve the first auxiliary stabilizer predisposed in the second channel 502, and carry the first auxiliary stabilizer to flow along the second channel 502. The first type of droplets in the first channel 501 and the continuous phase fluid dissolved with the first auxiliary stabilizer in the second channel 502 merge at the first confluence 106 to form the first liquid. The continuous phase fluid flowing into the fourth channel 504 can fully contact and dissolve the second auxiliary stabilizer predisposed in the fourth channel 504, and carry the second auxiliary stabilizer to flow along the fourth channel 504. The second type of droplets in the third channel 503 and the continuous phase fluid dissolved with the second auxiliary stabilizer in the fourth channel 504 merge at the second confluence 108 to form the second liquid. The first liquid flows along the first sub-channel 1091 of the connecting channel 109, the second liquid flows along the second sub-channel 1092 of the connecting channel 109, and finally the first liquid and the second liquid merge.

The first channel 501 has a first length L1, the second channel 502 has a second length L2, the third channel 503 has a third length L3, and the fourth channel 504 has a fourth length L4. In some embodiments, the first length L1 is less than or equal to the second length L2, and the third length L3 is less than or equal to the fourth length L4.

The first cross-section S1 of the first channel 501 has a first width W1 in a first direction, which is perpendicular to the flow direction F1 of the first type of droplets in the first channel 501; the second cross-section S2 of the second channel 502 has a second width W2 in a second direction, which is perpendicular to the flow direction F2 of the continuous phase fluid in the second channel 502; the third cross-section S3 of the third channel 503 has a third width W3 in a third direction, which is perpendicular to the flow direction F3 of the second type of droplets in the third channel 503; the fourth cross-section S4 of the fourth channel 504 has a fourth width W4 in a fourth direction, which is perpendicular to the flow direction F4 of the continuous phase fluid in the fourth channel 504. In some embodiments, the first width W1 is larger than the particle size of each of the first type of droplets so as to allow the first type of droplets to pass through, and the second width W2 is smaller than the particle size of each of the first type of droplets so as to not allow the first type of droplets to pass through. With such a design, the first type of droplets can be further prevented from flowing into the second channel 502, and the first type of droplets can be better separated from the first auxiliary stabilizer in the second channel 502, avoiding the first auxiliary stabilizer from affecting the stability of the first type of droplets. Similarly, the third width W3 is larger than the particle size of each of the second type of droplets so as to allow the second type of droplets to pass through, and the fourth width W4 is smaller than the particle size of each of the second type of droplets so as to not allow the second type of droplets to pass through. With such a design, the second type of droplets can be further prevented from flowing into the fourth channel 504, and the second type of droplets can be better separated from the second auxiliary stabilizer in the fourth channel 504, avoiding the second auxiliary stabilizer from affecting the stability of the second type of droplets.

In addition to having all the technical effects of the shunt structure 200, the shunt structure 500 can allow different types of droplets to flow through the first channel 501 and the third channel 503 by additionally arranging the third channel 503 and the fourth channel 504, and different auxiliary stabilizers are predisposed in the second channel 502 and the fourth channel 504 for different types of droplets, so as to improve the stability of different types of droplets in a targeted way.

FIG. 11 illustrates a schematic structural diagram of a microfluidic chip 6000. The microfluidic chip 6000 illustrated in FIG. 11 has basically the same structure as the microfluidic chip 1000 illustrated in FIG. 1 except that the shunt structure 600 is different, and therefore the same reference numerals are used to refer to the same components. For example, the microfluidic chip 6000 also comprises structures such as the first container 101, the second container 102, the delivery channel 103 (comprising the intersection 104), and the collection unit 105. Therefore, for the detailed structures and functions of components with the same reference numerals in FIG. 11 as those in FIG. 1, reference may be made to the description of FIG. 1, and details will not be repeated here. For brevity, only the differences are described below.

As illustrated in FIG. 11, the shunt structure 600 comprises a first channel 601, a second channel 602 and an auxiliary channel 603 communicating with the second channel 602. The auxiliary channel 603 is located between the second channel 602 and the first confluence 106, and the first channel 601 and the auxiliary channel 603 merge at the first confluence 106. The first channel 601 has a first cross-section S1, the second channel 602 has a second cross-section S2, and the area of the first cross-section S1 is larger than the area of the second cross-section S2. According to the correlation between the flow velocity and the channel width, the flow velocity of the first channel 601 is greater than the flow velocity of the second channel 602. Therefore, the droplets pass through the first channel 601, the continuous phase fluid passes through the second channel 602, and the auxiliary stabilizer can be predisposed in the second channel 602.

The upper right of FIG. 11 illustrates an enlarged view of the auxiliary channel 603. As illustrated, the auxiliary channel 603 has a variable width in a fifth direction, which is perpendicular to the flow direction F5 of the continuous phase fluid in the auxiliary channel 603. Specifically, the auxiliary channel 603 comprises alternately arranged first sections Q1 and second sections Q2, the first section Q1 has a fifth width W5 in the fifth direction, and the second section Q2 has a sixth width W6 in the fifth direction. The fifth width W5 is smaller than the sixth width W6. The width of the auxiliary channel 603 presents a style of “shrink-expand-shrink-expand”.

In addition to having all the technical effects of the shunt structure 200, according to the correlation between the flow velocity and the width of the channel, the flow velocity in the auxiliary channel 603 of the shunt structure 600 will change with the continuous change of the width, which will help to further improve the sufficient mixing of the continuous phase fluid and the auxiliary stabilizer, and is beneficial to further promote the sufficient dissolution of the auxiliary stabilizer.

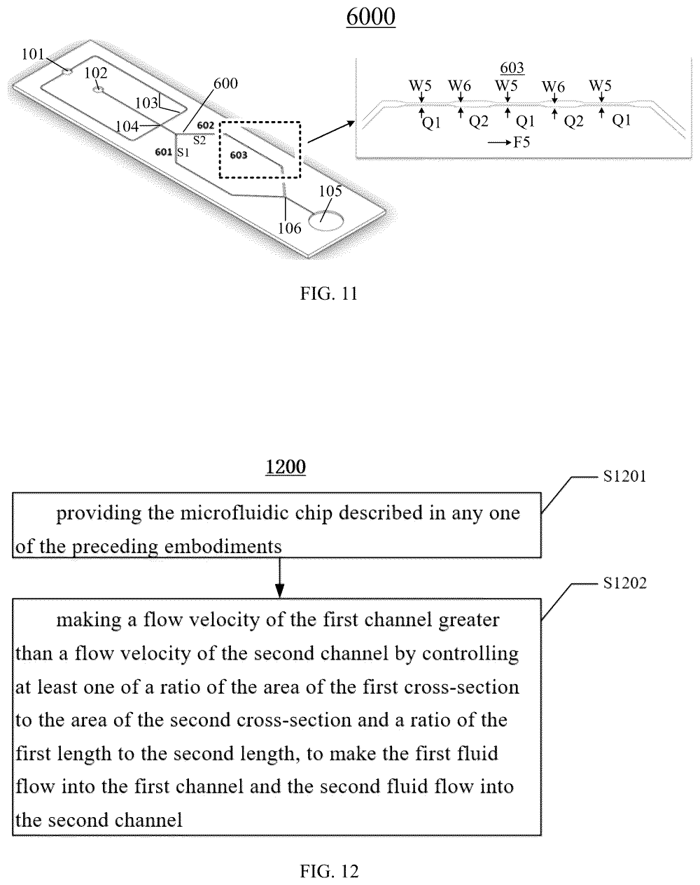

According to another aspect of the present disclosure, a method of controlling the flow velocity of fluid in a microfluidic chip is provided. FIG. 12 illustrates a flowchart of a method 1200, and the method 1200 comprises the following steps:
S1201: providing the microfluidic chip described in any one of the preceding embodiments;
S1202: making a flow velocity of the first channel greater than a flow velocity of the second channel by controlling at least one of a ratio of the area of the first cross-section S1 of the first channel of the shunt structure to the area of the second cross-section S2 of the second channel of the shunt structure and a ratio of the first length L1 of the first channel to the second length L2 of the second channel, to make the first fluid flow into the first channel and the second fluid flow into the second channel.

In some embodiments, the making a flow velocity of the first channel greater than a flow velocity of the second channel by controlling at least one of a ratio of the area of the first cross-section S1 of the first channel of the shunt structure to the area of the second cross-section S2 of the second channel of the shunt structure and a ratio of the first length L1 of the first channel to the second length L2 of the second channel, comprises: controlling the area of the first cross-section S1 to be equal to the area of the second cross-section S2 and the first length L1 to be smaller than the second length L2, such that the relationship between a first ratio of the second flow velocity V2 of the second channel to a first flow velocity V1 of the first channel and a second ratio of the first length L1 to the second length L2 is substantially linear. For details about the linear relationship, reference may be made to the description about FIG. 5, which will not be repeated here.

In some embodiments, the making a flow velocity of the first channel greater than a flow velocity of the second channel by controlling at least one of a ratio of the area of the first cross-section S1 of the first channel of the shunt structure to the area of the second cross-section S2 of the second channel of the shunt structure and a ratio of the first length L1 of the first channel to the second length L2 of the second channel, comprises: arranging the shape of the first cross-section S1 to be circular and the first cross-section S1 to have a first diameter D1, arranging the shape of the second cross-section S2 to be circular and the second cross-section S2 to have a second diameter D2, and controlling the first length L1 to be equal to the second length L2 and the first diameter D1 to be greater than the second diameter D2, such that a relationship between a first ratio of a second flow velocity V2 of the second channel to a first flow velocity V1 of the first channel and a third ratio of a square of the second diameter D2 to a square of the first diameter D1 is substantially linear. For details about the linear relationship, reference may be made to the description about FIG. 6, which will not be repeated here.

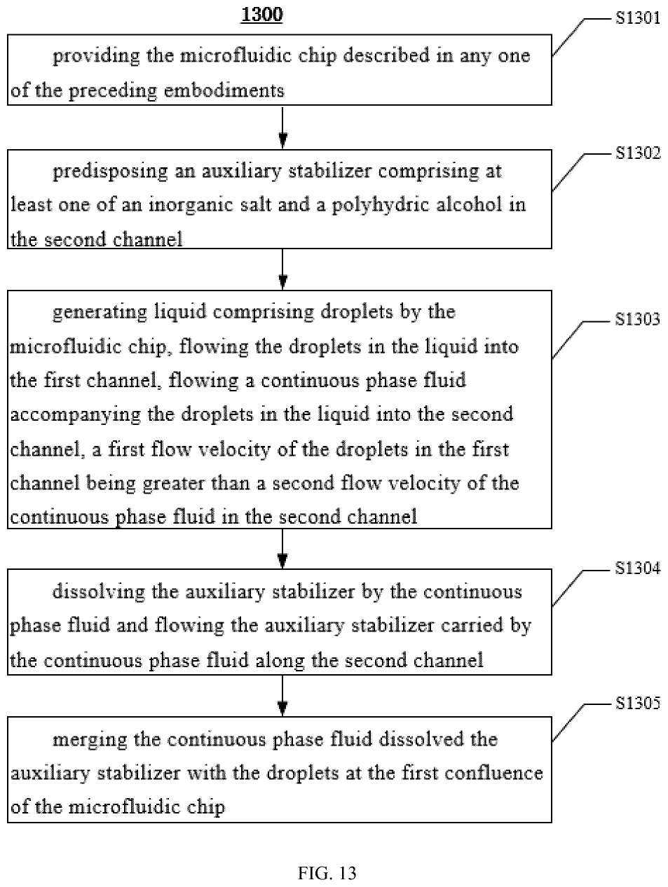

According to yet another aspect of the present disclosure, a method for using a microfluidic chip is provided. FIG. 13 illustrates a flowchart 1300 of the method, and the method 1300 comprises the following steps:
S1301: providing the microfluidic chip described in any of the previous embodiments;
S1302: predisposing an auxiliary stabilizer in the second channel, where the auxiliary stabilizer comprises at least one of an inorganic salt and a polyhydric alcohol;
S1303: generating liquid comprising droplets by the microfluidic chip, flowing the droplets in the liquid into the first channel, flowing a continuous phase fluid accompanying the droplets in the liquid into the second channel, a first flow velocity of the droplets in the first channel being greater than a second flow velocity of the continuous phase fluid in the second channel;
S1304: dissolving the auxiliary stabilizer by the continuous phase fluid and flowing the auxiliary stabilizer carried by the continuous phase fluid along the second channel; and
S1305: merging the continuous phase fluid dissolved the auxiliary stabilizer with the droplets at the first confluence of the microfluidic chip.

The polyhydric alcohol can be a variety of suitable materials, comprising but not limited to, ethanol, n-butanol, isobutanol, n-pentanol, sorbitol, glycerin. The inorganic salt can be any suitable material, comprising but not limited to, sodium chloride, magnesium sulfate, calcium chloride.

The above method 1300 can be applied to the microfluidic chip described in any of the above embodiments, for example, the method of using the microfluidic chip 1000 and the microfluidic chip 3000 may be exactly the same as the method 1300. The technical effects of the method of using the microfluidic chip 1000 and the microfluidic chip 3000 may be the same as those of the microfluidic chip 1000 and the microfluidic chip 3000, and for the sake of brevity, details are not repeated here.

The specific usage methods of several other microfluidic chips are introduced below with several examples.

The method 1400 for using the microfluidic chip 4000 illustrated in FIG. 9 may comprise the following steps:
S1401: providing the microfluidic chip 4000;
S1402: predisposing a first auxiliary stabilizer and a second auxiliary stabilizer different from the first auxiliary stabilizer in the second channel 402 and the third channel 403 respectively, the first auxiliary stabilizer comprising at least one of an inorganic salt and a polyhydric alcohol, and the second auxiliary stabilizer comprising at least one of an inorganic salt and a polyhydric alcohol;
S1403: generating the liquid by the microfluidic chip 4000, flowing the droplets in the liquid into the first channel 401, and flowing the continuous phase fluid accompanying the droplets in the liquid into the second channel 402 and the third channel 403 respectively, a first flow velocity of the droplets in the first channel 401 being greater than a second flow velocity of the continuous phase fluid in the second channel 402 and a third flow velocity of the continuous phase fluid in the third channel 403;
S1404: dissolving the first auxiliary stabilizer by the continuous phase fluid flowing into the second channel 402 and flowing the first auxiliary stabilizer carried by the continuous phase fluid along the second channel 402, dissolving the second auxiliary stabilizer by the continuous phase fluid flowing into the third channel 403 and flowing the second auxiliary stabilizer carried by the continuous phase fluid along the third channel 403; and
S1405: merging the continuous phase fluid dissolved the first auxiliary stabilizer, the continuous phase fluid dissolved the second auxiliary stabilizer, and the droplets at the first confluence 106.

The technical effect of the method 1400 can refer to the technical effect of the microfluidic chip 4000, and for the sake of brevity, details are not repeated here.

The method 1500 of using the microfluidic chip 5000 illustrated in FIG. 10 may comprise the following steps:
S1501: providing the microfluidic chip 5000;
S1502: predisposing a first auxiliary stabilizer and a second auxiliary stabilizer different from the first auxiliary stabilizer in the second channel 502 and the fourth channel 504 respectively, the first auxiliary stabilizer comprising at least one of an inorganic salt and a polyhydric alcohol, the second auxiliary stabilizer comprising at least one of an inorganic salt and a polyhydric alcohol;
S1503: generating liquid by the microfluidic chip 5000, the liquid comprising a first type of droplets, a second type of droplets and a continuous phase fluid accompanying the first type of droplets and the second type of droplets;
S1504: detecting in real time the liquid generated by the microfluidic chip 5000 at the sorting channel 110 by a detection device;

S1505: in response to detecting the first type of droplets, flowing the first type of droplets and the continuous phase fluid accompanying the first type of droplets into the first branch 1101 of the sorting channel 110 by applying an external force, flowing the first type of droplets into the first channel 501 through the first branch 1101, and flowing the continuous phase fluid accompanying the first type of droplets into the second channel 502 through the first branch 1101, the first flow velocity of the first type of droplets in the first channel 501 being greater than the second flow velocity of the continuous phase fluid in the second channel 502;
S1506: in response to detecting the second type of droplets, flowing the second type of droplets and the continuous phase fluid accompanying the second type of droplets into the second branch 1102 of the sorting channel 110 by applying an external force, flowing the second type of droplets into the third channel 503 through the second branch 1102, and flowing the continuous phase fluid accompanying the second type of droplets into the fourth channel 504 through the second branch 1102, the third flow velocity of the second type of droplets in the third channel 503 being greater than the fourth flow velocity of the continuous phase fluid in the fourth channel 504;
S1507: dissolving the first auxiliary stabilizer by the continuous phase fluid flowing into the second channel 502 and flowing the first auxiliary stabilizer carried by the continuous phase fluid along the second channel 502, and dissolving the second auxiliary stabilizer by the continuous phase fluid flowing into the fourth channel 504 and flowing the second auxiliary stabilizer carried by the continuous phase fluid along the fourth channel 504; and
S1508: merging the continuous phase fluid dissolved the first auxiliary stabilizer and the first type of droplets at the first confluence 106 to form a first liquid, merging the continuous phase fluid dissolved the second auxiliary stabilizer and the second type of droplets at the second confluence 108 to form a second liquid, and merging the first liquid and the second liquid via the connecting channel 109.

The technical effect of the method 1500 can refer to the technical effect of the microfluidic chip 5000, and for the sake of brevity, details are not repeated here.

The method 1600 of using the microfluidic chip 6000 illustrated in FIG. 11 may comprise the following steps:
S1601: providing the microfluidic chip 6000;
S1602: predisposing an auxiliary stabilizer in the second channel 602, where the auxiliary stabilizer comprising at least one of an inorganic salt and a polyhydric alcohol;
S1603: generating liquid comprising droplets by the microfluidic chip, flowing the droplets in the liquid into the first channel 601, flowing the continuous phase fluid accompanying the droplets in the liquid into the second channel 602, the first flow velocity of the droplets in the first channel 601 being greater than the second flow velocity of the continuous phase fluid in the second channel 602;
S1604: dissolving the auxiliary stabilizer by the continuous phase fluid and flowing the auxiliary stabilizer carried by the continuous phase fluid along the second channel 602 and the auxiliary channel 603, and changing the flow velocity of the continuous phase fluid carrying the auxiliary stabilizer in the auxiliary channel 603 with the change of the width of the auxiliary channel 603; and
S1605: merging the continuous phase fluid dissolved with the auxiliary stabilizer and the droplets at the first confluence 106 of the microfluidic chip 6000.

The technical effect of the method 1600 can refer to the technical effect of the microfluidic chip 6000, and for the sake of brevity, details are not repeated here.

It will be understood that although the terms first, second, third, etc. may be used herein to describe various elements, components, regions, layers and/or portions, these elements, components, regions, layers and/or portions should not be limited by these terms. These terms are only used to distinguish an element, component, region, layer or portion from another region, layer or portion. Thus, a first element, component, region, layer or portion discussed above could be termed a second element, component, region, layer or portion without departing from the teachings of the present disclosure.

Spatially relative terms such as “row”, “column”, “below”, “above”, “left”, “right”, etc. may be used herein for ease of description to describe factors such as the relationship of an element or feature to another element(s) or feature(s) illustrated in the figures. It will be understood that these spatially relative terms are intended to encompass different orientations of the device in use or operation in addition to the orientation depicted in the figures. For example, if the device in the figures is turned over, elements described as “below” other elements or features would then be oriented “above” other elements or features. Thus, the exemplary term “below” can encompass both an orientation of above and below. The device may be otherwise oriented (rotated 90 degrees or at other orientations) and the spatially relative descriptors used herein are interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to limit the present disclosure. As used herein, the singular forms “a,” “an,” and “the” are intended to comprise the plural forms as well, unless the context clearly dictates otherwise. It will be further understood that the terms “comprise” and/or “include” when used in this specification designate the presence of stated features, integers, steps, operations, elements and/or parts, but do not exclude the presence or addition of one or more other features, integers, steps, operations, elements, components and/or groups thereof. As used herein, the term “and/or” comprises any and all combinations of one or more of the associated listed items. In the description of this specification, description with reference to the terms “an embodiment,” “another embodiment,” etc. means that a particular feature, structure, material, or characteristic described in connection with the embodiment is included in at least one embodiment of the present disclosure. In this specification, schematic representations of the above terms are not necessarily directed to the same embodiment or example. Furthermore, the particular features, structures, materials or characteristics described may be combined in any suitable manner in any one or more embodiments or examples. Furthermore, those skilled in the art may combine the different embodiments or examples as well as the features of the different embodiments or examples described in this specification without conflicting each other.

It will be understood that when an element or layer is referred to as being “on”, “connected to”, “coupled to”, or “adjacent to” another element or layer, it may be directly on, directly connected to, directly coupled to, or directly adjacent to another element or layer, or intervening elements or layers may be present. In contrast, when an element is referred to as being “directly on”, “directly connected to”, “directly coupled to”, “directly adjacent to” another element or layer, with no intervening elements or layers present. However, in no case should “on” or “directly on” be interpreted as requiring a layer to completely cover the layer below.

Embodiments of the disclosure are described herein with reference to schematic illustrations (and intermediate structures) of idealized embodiments of the disclosure. As such, variations to the shapes of the illustrations are to be expected, e.g., as a result of manufacturing techniques and/or tolerances. Accordingly, embodiments of the present disclosure should not be construed as limited to the particular shapes of the regions illustrated herein, but are to comprise deviations in shapes due, for example, to manufacturing. Thus, the regions illustrated in the figures are schematic in nature and their shapes are not intended to illustrate the actual shape of a region of a device and are not intended to limit the scope of the present disclosure.

Unless otherwise defined, all terms (comprising technical and scientific terms) used herein have the same meaning as commonly understood by one of ordinary skill in the art to which this disclosure belongs. It will be further understood that terms such as those defined in commonly used dictionaries should be construed to have meanings consistent with their meanings in the relevant art and/or the context of this specification, and will not be idealized or overly interpreted in a formal sense, unless expressly defined as such herein.

The above descriptions are merely specific embodiments of the present disclosure, but the protection scope of the present disclosure is not limited thereto. Any changes or substitutions that those skilled in the art can easily think of within the technical scope disclosed by the present disclosure, should be comprised within the protection scope of the present disclosure. Therefore, the protection scope of the present disclosure should be based on the protection scope of the claims.

1. A microfluidic chip comprising at least one shunt structure,
wherein each of the at least one shunt structure comprises at least two channels, the at least two channels comprise a first channel and a second channel, the first channel is configured to allow a first fluid to flow therein, the second channel is configured to allow a second fluid to flow therein, and the first fluid and the second fluid merge at a first confluence of the microfluidic chip, and
wherein the first channel has a first cross-section and a first length, the second channel has a second cross-section and a second length, the first cross-section is perpendicular to a flow direction of the first fluid in the first channel, the second cross-section is perpendicular to a flow direction of the second fluid in the second channel, an area of the first cross-section is greater than or equal to an area of the second cross-section, and the first length is less than or equal to the second length.

2. The microfluidic chip according to claim 1, wherein the area of the first cross-section is equal to the area of the second cross-section and the first length is less than the second length, and a relationship between a first ratio of a second flow velocity of the second channel to a first flow velocity of the first channel and a second ratio of the first length to the second length is substantially linear.

3. (canceled)

4. The microfluidic chip according to claim 1, wherein the first cross-section is in a shape of circular and has a first diameter, and the second cross-section is in a shape of circular and has a second diameter, the first length is equal to the second length and the first diameter is greater than the second diameter, and a relationship between a first ratio of a second flow velocity of the second channel to a first flow velocity of the first channel and a third ratio of a square of the second diameter to a square of the first diameter is substantially linear.

5. (canceled)

6. The microfluidic chip according to claim 1, wherein the area of the first cross-section is greater than the area of the second cross-section and the first length is less than the second length, the first channel and the second channel merge at the first confluence,
wherein the first cross-section has a first width in a first direction, the first direction is perpendicular to the flow direction of the first fluid in the first channel, the first fluid comprises droplets,
wherein the second cross-section has a second width in a second direction, the second direction is perpendicular to the flow direction of the second fluid in the second channel, and
wherein the first width is larger than a particle size of each of the droplets, and the second width is smaller than the particle size of each of the droplets.

7. (canceled)

8. (canceled)

9. The microfluidic chip according to claim 1, wherein the area of the first cross-section is equal to the area of the second cross-section and the first length is less than the second length, the first channel and the second channel merge at the first confluence,
wherein the first channel comprises at least one section, each of the at least one section is in a shape of S-shaped,
wherein the second channel comprises at least one section, each of the at least one section is in a shape of reverse S-shaped, and
wherein a number of the sections of the second channel is same as a number of the sections of the first channel, and a length of each section of the second channel is greater than a length of each section of the first channel.

10. (canceled)

11. (canceled)

12. The microfluidic chip according to claim 1, wherein each shunt structure further comprises a third channel configured to allow the second fluid to flow therein, the first channel, the second channel and the third channel merge at the first confluence,
wherein the third channel has a third cross-section and a third length, the third cross-section is perpendicular to a flow direction of the second fluid in the third channel, the area of the first cross-section, the area of the second cross-section and an area of the third cross-section are equal, and the first length is less than the second length and the third length, and
wherein the first channel is between the second channel and the third channel, and the second channel and the third channel are axisymmetric with respect to the first channel.

13. (canceled)

14. The microfluidic chip according to claim 1, wherein each shunt structure further comprises:
a third channel having a third cross-section and configured to allow a third fluid to flow therein, the third cross-section being perpendicular to a flow direction of the third fluid in the third channel;
a fourth channel having a fourth cross-section and configured to allow a fourth fluid to flow therein, the fourth cross-section being perpendicular to a flow direction of the fourth fluid in the fourth channel, the third channel and the fourth channel merging at a second confluence of the microfluidic chip; and
a connecting channel communicating with the first confluence and the second confluence respectively,
wherein the area of the first cross-section is greater than the area of the second cross-section, and an area of the third cross-section is greater than an area of the fourth cross-section, and
wherein the third channel has a third length, the fourth channel has a fourth length, the first length is less than or equal to the second length, and the third length is less than or equal to the fourth length.

15. (canceled)

16. The microfluidic chip according to claim 14, wherein the first cross-section has a first width in a first direction, the first direction is perpendicular to the flow direction of the first fluid in the first channel,
wherein the second cross-section has a second width in a second direction, the second direction is perpendicular to the flow direction of the second fluid in the second channel,
wherein the third cross-section has a third width in a third direction, the third direction is perpendicular to the flow direction of the third fluid in the third channel,
wherein the fourth cross-section has a fourth width in a fourth direction, the fourth direction is perpendicular to the flow direction of the fourth fluid in the fourth channel, and
wherein the first fluid comprises a first type of droplets, the third fluid comprises a second type of droplets, the first width is greater than a particle size of each of the first type of droplets and the second width is less than the particle size of each of the first type of droplets, the third width is greater than a particle size of each of the second type of droplets and the fourth width is less than the particle size of each of the second type of droplets.

17. The microfluidic chip according to claim 14, further comprising a sorting channel located upstream of the shunt structure, wherein the sorting channel comprises a first branch and a second branch, the first branch communicates with the first channel and the second channel, and the second branch communicates with the third channel and the fourth channel.

18. The microfluidic chip according to claim 1, wherein each shunt structure further comprises an auxiliary channel communicated with the second channel, the auxiliary channel is between the second channel and the first confluence, and the first channel and the auxiliary channel merge at the first confluence, the area of the first cross-section is greater than the area of the second cross-section, the auxiliary channel has a variable width in a fifth direction, the fifth direction is perpendicular to a flow direction of the second fluid in the auxiliary channel, and
wherein the auxiliary channel comprises a first section and a second section which are alternately arranged, the first section has a fifth width in the fifth direction, the second section has a sixth width in the fifth direction, the fifth width is smaller than the sixth width.

19. (canceled)

20. The microfluidic chip according to claim 1, wherein the at least one shunt structure is a plurality of shunt structures, and the plurality of shunt structures are arranged at intervals from each other.

21. The microfluidic chip according to claim 1, further comprising:
a droplet generation unit located upstream of the shunt structure and communicating with the shunt structure; and
a collection unit located downstream of the shunt structure and communicating with the shunt structure.

22. (canceled)

23. A method of controlling a flow velocity of a fluid in a microfluidic chip, comprising:
providing the microfluidic chip according to claim 1; and
making a flow velocity of the first channel greater than a flow velocity of the second channel by controlling at least one of a ratio of the area of the first cross-section to the area of the second cross-section and a ratio of the first length to the second length, to make the first fluid flow into the first channel and the second fluid flow into the second channel.

24. The method according to claim 23, wherein the making a flow velocity of the first channel greater than a flow velocity of the second channel by controlling at least one of a ratio of the area of the first cross-section to the area of the second cross-section and a ratio of the first length to the second length, comprises:
controlling the area of the first cross-section to be equal to the area of the second cross-section and the first length to be less than the second length, such that a relationship between a first ratio of a second flow velocity of the second channel to a first flow velocity of the first channel and a second ratio of the first length to the second length is substantially linear.

25. The method according to claim 23, wherein the making a flow velocity of the first channel greater than a flow velocity of the second channel by controlling at least one of a ratio of the area of the first cross-section to the area of the second cross-section and a ratio of the first length to the second length, comprises:
arranging a shape of the first cross-section to be circular and the first cross-section to have a first diameter, arranging a shape of the second cross-section to be circular and the second cross-section to have a second diameter, controlling the first length to be equal to the second length and the first diameter to be greater than the second diameter, such that a relationship between a first ratio of a second flow velocity of the second channel to a first flow velocity of the first channel and a third ratio of a square of the second diameter to a square of the first diameter is substantially linear.

26. A method of using a microfluidic chip, comprising:
providing the microfluidic chip according to claim 1;
predisposing an auxiliary stabilizer comprising at least one of an inorganic salt and a polyhydric alcohol in the second channel;
generating liquid comprising droplets by the microfluidic chip, flowing the droplets in the liquid into the first channel, flowing a continuous phase fluid accompanying the droplets in the liquid into the second channel, a first flow velocity of the droplets in the first channel being greater than a second flow velocity of the continuous phase fluid in the second channel;
dissolving the auxiliary stabilizer by the continuous phase fluid and flowing the auxiliary stabilizer carried by the continuous phase fluid along the second channel; and
merging the continuous phase fluid dissolved the auxiliary stabilizer with the droplets at the first confluence of the microfluidic chip.

27. The method according to claim 26, wherein each shunt structure further comprises a third channel, the first channel, the second channel and the third channel merge at the first confluence of the microfluidic chip, the third channel has a third cross-section and a third length, the third cross-section is perpendicular to a flow direction of the fluid in the third channel, the area of the first cross-section, the area of the second cross-section and an area of the third cross-section are equal, and the first length is less than the second length and the third length,
wherein, the method comprises:
predisposing a first auxiliary stabilizer and a second auxiliary stabilizer different from the first auxiliary stabilizer in the second channel and the third channel respectively, the first auxiliary stabilizer comprising at least one of an inorganic salt and a polyhydric alcohol, and the second auxiliary stabilizer comprising at least one of an inorganic salt and a polyhydric alcohol;
generating the liquid by the microfluidic chip, flowing the droplets in the liquid into the first channel, and flowing the continuous phase fluid accompanying the droplets in the liquid into the second channel and the third channel respectively, a first flow velocity of the droplets in the first channel being greater than a second flow velocity of the continuous phase fluid in the second channel and a third flow velocity of the continuous phase fluid in the third channel;
dissolving the first auxiliary stabilizer by the continuous phase fluid flowing into the second channel and flowing the first auxiliary stabilizer carried by the continuous phase fluid along the second channel, dissolving the second auxiliary stabilizer by the continuous phase fluid flowing into the third channel and flowing the second auxiliary stabilizer carried by the continuous phase fluid along the third channel; and
merging the continuous phase fluid dissolved the first auxiliary stabilizer, the continuous phase fluid dissolved the second auxiliary stabilizer, and the droplets at the first confluence.

28. The method according to claim 26, wherein each shunt structure further comprises a third channel, a fourth channel and a connecting channel, the third channel and the fourth channel merge at a second confluence of the microfluidic chip, the connecting channel communicates with the first confluence and the second confluence respectively, the third channel has a third cross-section, the fourth channel has a fourth cross-section, the third cross-section is perpendicular to a flow direction of the fluid in the third channel, the fourth cross-section is perpendicular to a flow direction of the fluid in the fourth channel, the area of the first cross-section is greater than the area of the second cross-section, and an area of the third cross-section is greater than an area of the fourth cross-section,
wherein the method comprises:
predisposing a first auxiliary stabilizer and a second auxiliary stabilizer different from the first auxiliary stabilizer in the second channel and the fourth channel respectively, the first auxiliary stabilizer comprising at least one of an inorganic salt and a polyhydric alcohol, the second auxiliary stabilizer comprising at least one of an inorganic salt and a polyhydric alcohol;
generating liquid by the microfluidic chip, the liquid comprising a first type of droplets, a second type of droplets and a continuous phase fluid accompanying the first type of droplets and the second type of droplets, the first type of droplets flowing into the first channel, the second type of droplets flowing into the third channel, the continuous phase fluid flowing into the second channel and the fourth channel respectively, a first flow velocity of the first type of droplets in the first channel being greater than a second flow velocity of the continuous phase fluid in the second channel, a third flow velocity of the second type of droplets in the third channel being greater than a fourth flow velocity of the continuous phase fluid in the fourth channel;
dissolving the first auxiliary stabilizer by the continuous phase fluid flowing into the second channel and flowing the first auxiliary stabilizer carried by the continuous phase fluid along the second channel, and dissolving the second auxiliary stabilizer by the continuous phase fluid flowing into the fourth channel and flowing the second auxiliary stabilizer carried by the continuous phase fluid along the fourth channel; and
merging the continuous phase fluid dissolved the first auxiliary stabilizer and the first type of droplets at the first confluence to form a first liquid, merging the continuous phase fluid dissolved the second auxiliary stabilizer and the second type of droplets at the second confluence to form a second liquid, and merging the first liquid and the second liquid via the connecting channel.

29. The method according to claim 28, wherein the microfluidic chip further comprises a sorting channel located upstream of the shunt structure, the sorting channel comprises a first branch and a second branch, the first branch communicates with the first channel and the second channel, the second branch communicates with the third channel and the fourth channel,
wherein the generating liquid by the microfluidic chip comprises:
detecting in real time the liquid generated by the microfluidic chip at the sorting channel by a detection device;
in response to detecting the first type of droplets, flowing the first type of droplets and the continuous phase fluid accompanying the first type of droplets into the first branch of the sorting channel by applying an external force, flowing the first type of droplets into the first channel through the first branch, and flowing the continuous phase fluid accompanying the first type of droplets into the second channel through the first branch; and
in response to detecting the second type of droplets, flowing the second type of droplets and the continuous phase fluid accompanying the second type of droplets into the second branch of the sorting channel by applying an external force, flowing the second type of droplets into the third channel through the second branch, and flowing the continuous phase fluid accompanying the second type of droplets into the fourth channel through the second branch.

30. The method according to claim 26, wherein each shunt structure further comprises an auxiliary channel communicated with the second channel, the auxiliary channel is between the second channel and the first confluence, the first channel and the auxiliary channel merge at the first confluence, the area of the first cross-section is greater than the area of the second cross-section, the auxiliary channel has a variable width in a fifth direction, the fifth direction is perpendicular to a flow direction of the continuous phase fluid in the auxiliary channel, and
wherein the dissolving the auxiliary stabilizer by the continuous phase fluid and flowing the auxiliary stabilizer carried by the continuous phase fluid along the second channel, further comprises:
dissolving the auxiliary stabilizer by the continuous phase fluid and flowing the auxiliary stabilizer carried by the continuous phase fluid along the second channel and the auxiliary channel, and changing the flow velocity of the continuous phase fluid carrying the auxiliary stabilizer in the auxiliary channel with the change of the width of the auxiliary channel.

31. (canceled)
